(19) United States
(12) Patent Application Publication
(10) Pub. No.: US 2005/0080414 A1
(43) Pub. Date: Apr. 14, 2005

(54) Spinal fixation hooks and method of spinal fixation

(76) Inventors: Thomas R. Keyer, Aston, PA (US); Martin Walther, West Chester, PA (US)

Correspondence Address: JONES DAY, 51 Louisiana Aveue, N.W, WASHINGTON, DC, 20001-2113, US

(21) Appl. No.: 10/682,997

(22) Filed: Oct. 14, 2003

(51) Int. Cl. A61B 17/70; A61B 17/28

(52) U.S. Cl. 606/61; 606/207

(57) ABSTRACT

The present invention is directed to a spinal fixation device. The device may include a body defining a channel for receiving a spinal rod, the channel having a first branch and a second branch separated by a lateral opening, a hook extending from the body for engaging a portion of a vertebra, and a fastener configured to secure the spinal rod in the channel. The fastener may extend through only the first branch. The spinal fixation device may also include one or more tool engagement recesses. Additionally, the present invention is directed to methods of spinal fixation.

### TECHNICAL FIELD OF THE INVENTION

The present invention relates generally to spinal fixation devices and methods of spinal fixation. More specifically, the present invention relates to hooks for spinal fixation, and methods of spinal fixation using the same.

### BACKGROUND OF THE INVENTION

There are many known spinal conditions that require the imposition and/or maintenance of corrective forces on the spine in order to return the spine to its normal condition. As a result, numerous systems have been developed for use in spinal fixation. One type of spinal fixation system generally includes one or more spinal rods placed parallel to the spine with fixation devices (such as hooks, screws, or plates) interconnected between the spinal rods and selected portions of the spine. One problem with some known spinal fixation devices is that they usually require the surgeon to insert the spinal rod onto the devices in the axial direction (i.e., in series), making it difficult for the surgeon to subsequently add or remove the devices without disassembling all or a part of the system. In an attempt to alleviate this difficulty, devices have been developed that allow insertion of the spinal rod onto the devices in a lateral direction. Many of these devices, however, require insertion of the spinal rod onto the devices from the side (verses from the top or from an angle) and thus require a larger incision into the patient than is necessary or desirable. In addition, many of these devices require components such as screws to be completely removed from the devices in order to allow attachment of the spinal rod. Completely removing these components may increase the amount of time necessary for the procedure, and may also increase the risk of small parts being lost inside the patient. Many of the known devices also lack features that allow them to be easily grasped and manipulated with instruments.

Therefore, there remains a need in the art for spinal fixation devices that provide for minimally invasive implantation of the device and/or spinal rods, are capable of being easily and quickly connected and disconnected from the spinal rods, and are easy to grasp and manipulate with instruments.

### SUMMARY OF THE INVENTION

The present invention is directed to a spinal fixation device. The device may include a body defining a channel for receiving a spinal rod, the channel having a first branch and a second branch separated by a lateral opening, a hook extending from the body for engaging a portion of a vertebra, and a fastener configured to secure the spinal rod in the channel. The fastener may extend through only the first branch. The first branch may be an upper branch or a lower branch of the channel. The fastener may extend through a bore provided in the first branch. The fastener may include a head receivable in the bore and a tip opposite the head, and the tip may be spaced from the second branch when the fastener secures the spinal rod in the channel. The fastener may be a set screw, and at least one of the set screw and the bore may be threaded. The lateral opening is preferably configured to permit insertion of the spinal rod into the channel through the lateral opening without removing the fastener from the first branch. The tip of the fastener may engage the spinal rod to secure the spinal rod in the channel. For example, the tip may include a ramp surface that engages the spinal rod. Additionally or alternatively, the tip may be substantially conical or frustoconical. According to one aspect of the invention, the hook may define a first axis and the channel may define a second axis that is inclined with respect to the first axis by greater than about 90°, and preferably by between about 100° and about 135°. The hook may be configured and dimensioned to engage a lamina.

According to another aspect of the invention, the spinal fixation device may include a body defining a rod-receiving channel having a lateral opening, the body including at least one tool engagement recess for gripping by a tool, a hook extending from the body for engaging a portion of a vertebra, and a fastener configured to secure the spinal rod in the channel. The body may include a first side and a second side opposite the first side, and a first tool engagement recess may be formed on the first side and a second tool engagement recess may be formed on the second side. The first and second tool engagement recesses may be configured and dimensioned to engage first and second jaws of a forceps or other tool. The tool engagement recesses may be configured for gripping from above when the hook is engaged with a portion of a vertebra. Additionally or alternatively, the tool engagement recess may be configured to allow gripping with a forceps without the forceps blocking the lateral opening.

The present invention is also directed to a method of spinal fixation. The method may include the steps of providing a first spinal fixation device having a body with an open channel for receiving a spinal rod, a hook extending from the body, and a fastener engaged with the body and capable of partially blocking the open channel, engaging the hook to a portion of a first vertebra (such as the lamina), moving the spinal rod into the open channel without disengaging the fastener from the body, and tightening the fastener against the spinal rod to secure the spinal rod in the channel. The step of engaging the hook to a portion of the first vertebra may occur before or after the step of moving the spinal rod into the open channel. The step of moving the spinal rod into the open channel may comprise moving the spinal rod into the open channel, in which case the spinal rod may be moved upward into the open channel at an angle of between about 20° and about 70° with respect to a medial plane of a patient. Alternatively, the step of moving the spinal rod into the open channel may comprise moving the open channel toward the spinal rod, in which case the first spinal fixation device may be moved downward onto the spinal rod at an angle of between about 20° and about 70° with respect to a medial plane of a patient.

The method may further include the steps of attaching a second spinal fixation device to a portion of a second vertebra and securing the spinal rod to the second spinal fixation device. The step of attaching the second spinal fixation device may occurs before or after the steps of engaging the hook to a portion of the first vertebra and moving the spinal rod into the open channel. Additionally or alternatively, the method may further include the step of compressing or distracting the first vertebra with respect to the second vertebra, and/or aligning the first vertebra with respect to the second vertebra. The method may further include removing the spinal rod from the open channel without completely disengaging the fastener from the body.

### BRIEF DESCRIPTION OF THE DRAWINGS

The detailed description will be better understood in conjunction with the accompanying drawings, wherein like reference characters represent like elements, as follows:

FIG. 1 is a perspective view of a first illustrative embodiment of a spinal hook according to the present invention, shown attached to a spinal rod;

FIG. 2 is a side view of second illustrative embodiment of a spinal hook according to the present invention;

FIG. 3 is a top view of the spinal hook of FIG. 2;

FIG. 4 is an end view of the spinal hook of FIG. 2;

FIG. 5 is a side view of a first illustrative embodiment of a forceps for grasping the spinal hooks of the present invention;

FIG. 6 is a top view of the forceps of FIG. 5; and

FIG. 7 is a superior view of a cervical vertebra having a spinal hook according to the present invention attached thereto.

### DETAILED DESCRIPTION OF THE PREFERRED EMBODIMENTS

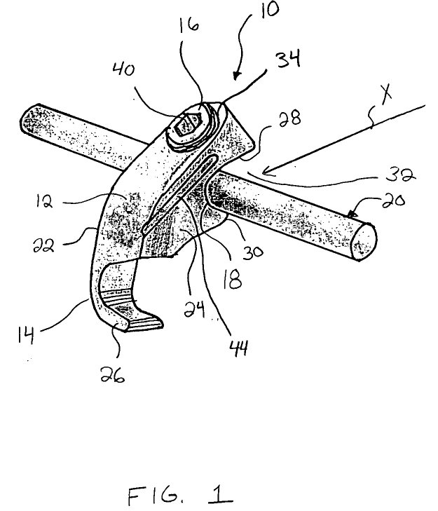

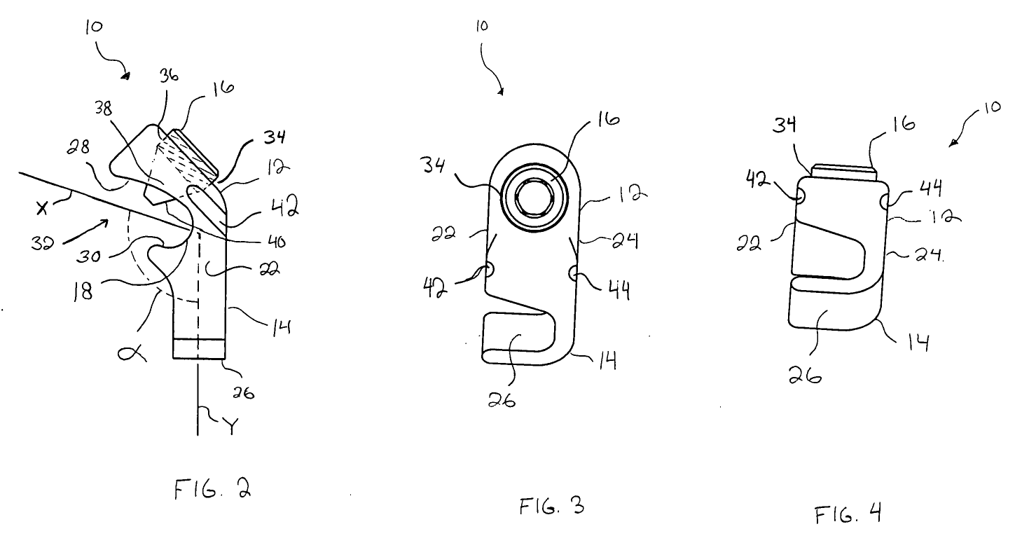

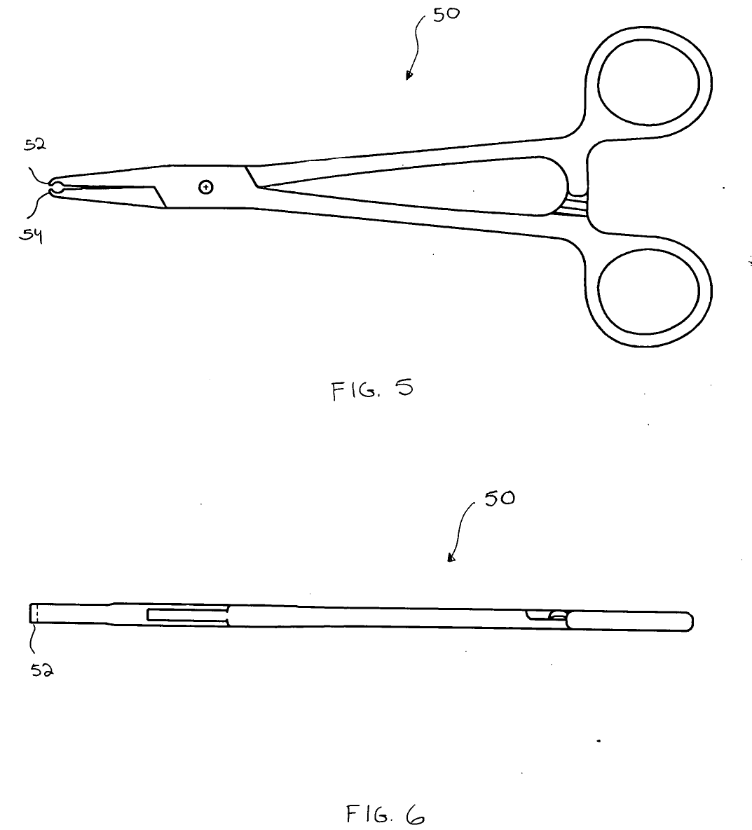

Referring to FIG. 1, a first illustrative embodiment of a spinal fixation device according to the present invention is shown attached to a spinal rod. The spinal fixation device, shown as spinal hook 10, generally includes a body 12, a hook 14 extending from the body 12, and a fastener 16 engaged with the body 12. Body 12 may define a rod-receiving channel 18 for receiving the spinal rod 20. In the illustrative embodiment of FIG. 1, hook 14 is attached at the left side of body 12 and extends to the right (as viewed in FIG. 1), and thus is called a “left” embodiment. The illustrative embodiment of FIGS. 2-4 is substantially identical to the embodiment of FIG. 1 except that it is a “right” embodiment, in that the hook 14 is attached at the right side of body 12 and extends to the left (as viewed in FIGS. 3 and 4). The right and left embodiments may be provided, for example, to allow spinal hooks 10 to be attached to a portion of a vertebra, such as a lamina, from both sides of the vertebra. The right and left embodiments may also be provided with their hooks 14 oriented toward one another in a claw configuration, although additional advantages of the right and left embodiments are contemplated and known by those of ordinary skill in the art. As the only significant structural difference between the embodiment of FIG. 1 and that of FIGS. 2-4 is the orientation of the hook 14 with respect to body 12, these two illustrative embodiments will be described together herein.

Referring now to FIGS. 1-4, body 12 defines a rod-receiving channel 18 for receiving spinal rod 20. Body 12 preferably has a low profile with smooth or rounded edges in order to be minimally invasive. While body 12 is shown in FIGS. 1-4 as generally rectangular, the present invention is not limited to any specific shape, size or configuration of body 12.

Spinal fixation device 10 is preferably configured and dimensioned for attachment to a portion of a vertebra in the cervical or upper thoracic regions of the spine, however spinal fixation device 10 may alternatively be configured and dimensioned for attachment to vertebrae in the other areas of the spine (i.e., the lower thoracic and lumbar regions). In the illustrative embodiments shown in FIGS. 1-4, hook 14 is configured and dimensioned to engage the underside of the lamina portion of a vertebra. For example, hook 14 may extend downward from the left side 22 of body 12 and curve toward the right side 24 (as shown in FIG. 1), or alternatively may extend downward from the right side 24 of body 12 and curve toward the left side 22 (as shown in FIGS. 2-4). In either configuration, hook 14 may terminate in a narrow blade portion 26 that easily fits under the lamina portion of the vertebra. However, one of ordinary skill in the art will know and understand from this disclosure that hook 14 may alternatively be configured and dimension to engage other parts of the vertebra such as, for example, the spinous processes or pedicles.

As shown in FIGS. 1 and 2, rod-receiving channel 18 may define a first branch 28 (shown for illustrative purposes as an upper branch) and a second branch 30 (shown for illustrative purposes as a lower branch) separated by a lateral opening 32. Opening 32 is preferably configured and dimensioned to allow insertion of spinal rod 20 into the rod-receiving channel 18 through opening 32 (verses conventional axial or lengthwise insertion), as generally indicated by the axis X of FIGS. 1 and 2. More specifically, the mouth of opening 32 may be larger than the diameter of spinal rod 20. This may facilitate easier attachment and removal of spinal hook 10 from spinal rod 20 without having to move or remove other spinal fixation devices already attached to spinal rod 20. As shown in FIGS. 1 and 2, first branch 28 is longer than second branch 30, however first and second branches 28 and 30 may alternatively be of equal length, or second branch 30 may be longer than first branch 28.

Still referring to FIGS. 1 and 2, fastener 16 may extend through a bore 34 in the first branch 28 and partially block opening 32, thereby allowing fastener 16 to secure spinal rod 20 in channel 18. Fastener 16 preferably extends through only the first branch 28 or the second branch 30 (and not both). This configuration in conjunction with a sufficiently large opening 32 may allow spinal rod 20 to be inserted through opening 32 without having to completely remove fastener 16 from bore 34. While first branch 28 is shown in FIGS. 1-4 as the upper branch, first branch 28 may alternatively be the lower branch, and fastener 16 may extend through the lower branch, as would be understood by one of ordinary skill in the art from this disclosure.

Referring to FIG. 2, fastener 16 (shown partially in phantom) may include a head portion 36 that engages bore 34 and a tip portion 38 that is opposite head portion 36 and extends into opening 32. Fastener 16 is shown as a set screw having threads on head portion 36 that mate with threads on bore 34, however other configurations known in the art are contemplated. For example, fastener 16 may alternatively be configured as a quarter-turn cam. Tip portion 38 may include an inclined or ramp surface 40 that engages spinal rod 20 to press spinal rod 20 into channel 18 and secure spinal rod 20 to body 12 (when fastener 16 is sufficiently tightened in bore 34). As shown, tip portion 38 may be substantially conical or frustoconical, however other configurations are possible. It may be preferable that tip portion 38 is spaced from second branch 30 when the spinal rod 20 is secured in rod-receiving channel 18, thereby further facilitating insertion and/or removal of spinal rod 20 from channel 18 through opening 32 without having to completely remove fastener 16 from bore 34. As shown in FIG. 1, fastener 16 may include an internal socket 40 for receiving a driving tool, such as a hex wrench or a torx wrench. Alternatively, fastener 16 may have an external feature, such as a hex-shaped protrusion for engagement by a socket wrench or other tool.

Referring to FIGS. 1 and 2, rod-receiving channel 18 and/or body 12 may be angled upward with respect to hook 14 to allow spinal rod 20 to be inserted into rod-receiving channel 18 through opening 32 from the top and/or side when hook 14 is implanted in the patient. This may also allow spinal hook 10 to be attached onto a spinal rod that is already implanted by moving spinal hook 10 downward/sideways with respect to the rod. This top or angled loading may reduce the size of the incision required for implantation of the spinal hook 10 and/or spinal rod 20, and may also provide simplified implantation as well as other benefits. As shown in FIG. 2, hook 14 may generally define a first axis Y and rod-receiving channel 18 may define a second axis X. Second axis X may be inclined with respect to first axis Y by an angle α of greater than about 90°. Preferably, angle α is between about 100° and about 135°. One of ordinary skill in the art will know and appreciate, however, that other values of angle α are possible.

Referring to FIGS. 1-4, body 12 may include one or more tool engagement recesses for gripping with a tool, such as the forceps 50 shown in FIGS. 5 and 6. As shown, a first tool engagement recess 42 may be formed on the left side 22 of body 12, and a second tool engagement recess 44 may be formed on the right side 24 of body 12. The recesses 42, 44 may be elongated slots formed on body 12, in which case recesses 42, 44 may receive the jaws 52, 54 of forceps 50, thereby allowing spinal hook 10 to be implanted and/or manipulated using forceps 50. Recesses 42, 44 preferably extend from the outer peripheral surface of body 12 toward fastener 16, however other configurations are contemplated. It should be noted that recesses 42, 44 and jaws 52, 54 are not limited to the elongated configurations shown in the figures. Rather, recesses 42, 44 and jaws 52, 54 may have any mating configurations that allow forceps 50 to securely grasp body 12, as would be appreciated by one of ordinary skill in the art from this disclosure. When used with an instrument such as forceps 50, recesses 42, 44 may provide sufficient grip on body 12 for a surgeon to maneuver spinal fixation device 10 into position through obstructions such as ligaments and other tissues. Preferably, recesses 42, 44 are configured and dimensioned (e.g. are long and deep enough) to allow a surgeon to use forceps 50 to maneuver spinal fixation device 10 into engagement with the lamina through the surrounding ligaments.

Additionally, recesses 42, 44 may be positioned (e.g., as shown in FIGS. 1-4) to allow the surgeon to grip spinal fixation device 10 from above, to provide for maneuvering of spinal fixation device 10 into position on the vertebra from above, or outside of the patient. It is also preferred that recesses 42, 44 are positioned at a sufficient distance or orientation with respect to the opening 32, to allow the rod 20 to be inserted into channel 18 through opening 32 while recesses 42, 44 are engaged by an instrument. In other words, recesses 42, 44 are preferably not located in a position in which the instrument, for example forceps 50, blocks access to the opening 32 when forceps 50 are engaged with recesses 42, 44. Recesses 42, 44 are also preferably located in a position in which the instrument, such as forceps 50, does not block access to the fastener 16 when the instrument is grasping the recesses 42, 44.

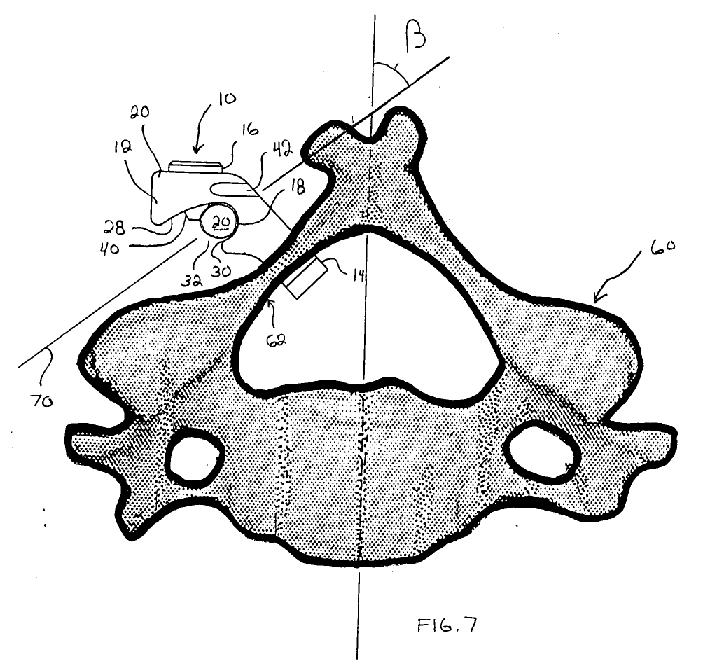

The present invention is also directed to a method of spinal fixation, which will now be described with reference to the structures described above, although the inventive method is not limited to the described structures. The method includes preparing an incision in the patient to provide access to a preselected region of the spine. Preferably, the incision is sufficient to allow implantation of one or more spinal rods. Once the incision has been prepared and the spine is ready for implantation, a first spinal hook 10 may be engaged to a portion of a first vertebra 60, as shown in FIG. 7. Preferably, the hook 14 is engaged underneath the lamina 62 of the first vertebra 60, although it may alternatively be engaged to other regions of the vertebra, such as the spinous processes or pedicles. Hook 14 is engaged to the lamina 62 of a cervical vertebra in FIG. 7, however the method may have application to other regions of the spine, such as the thoracic and lumbar regions. A second spinal fixation device (not shown), such as a second spinal hook 10 (or alternatively a pedicle screw or other known spinal fixation device) may be engaged to a portion of a second vertebra (not shown). One of ordinary skill in the art will know that the second spinal fixation device may be implanted before or after the first spinal hook 10. One of ordinary skill in the art will also know that any number of spinal fixation devices may be implanted as may be required by a specific application and/or disorder.

The spinal rod 20 may be attached to the first spinal hook 10 and the second spinal fixation device. This step may occur before or after the first spinal hook 10 and/or the second spinal fixation device is engaged to the portion of the vertebra. That is, the first spinal hook 10 and/or the second spinal fixation device may be implanted before a spinal rod, or alternatively, the first spinal hook 10 and/or the second spinal fixation device may be implanted and attached to a spinal rod that is already implanted and anchored to the spinal column.

With respect to at least the first spinal hook 10, the spinal rod may be moved into channel 18 through opening 32, such as in the direction 70 of FIG. 7. For example, this may include moving the rod 20 upward into the channel 18 at an angle β of between about 20° and about 70° with respect to the medial plane P of the patient (the plane dividing the human body into right and left halves). Preferably, this may include moving the rod 20 upward at an angle β of between about 30° and about 60° with respect to the medial plane P, although other angles are possible. Alternatively, first spinal hook 10 may be inserted downward onto spinal rod 20 until spinal rod 20 enters channel 18, such as in the case where spinal hook 10 is being added to a spinal rod construct that is already in place on the spinal column. This may include moving the first spinal hook 10 downward toward spinal rod 20 at an angle β of between about 20° and about 70° with respect to the medial plane P of the patient. Preferably, this may include moving the rod 20 downward at an angle β of between about 30° and about 60° with respect to the medial plane P, although other angles are possible.

The spinal rod 20 is preferably inserted into the rod-receiving channel 18 without disengaging or removing the fastener 16 from body 12. This may be possible, for example, due to the configuration in which fastener 16 extends through only one of the first and second branches 28, 30 of the rod-receiving channel 18, although other structures may make this procedure possible.

Corrective forces may be applied to at least the first and second vertebrae to correct the disorder at hand. These corrective forces may be applied before or after the first spinal hook 10 and/or the second spinal fixation device are attached to the spine, or before the rod 20 is secured to these devices. A compression or distraction tool may be used to move the first and second vertebrae towards or away from one another, or alternatively, forces may be applied to the vertebrae to bring them into proper alignment (e.g., to correct abnormal curvature of the spine). The first spinal hook 10 and/or the second spinal fixation device may be secured to the spinal rod to maintain those desired positions. With respect to at least the first spinal hook 10, this may include tightening the fastener 16 against the spinal rod 20, which may cause inclined surface 40 of fastener 16 to bias the spinal rod 20 into rod-receiving channel 18.

The method may also include the step of removing first spinal hook 10 and/or another spinal fixation device attached to the spinal rod. In the case of at least first spinal hook 10, this may be accomplished by moving the spinal rod through opening 32 in rod-receiving channel 18. This may be possible without removing or disengaging fastener 16 from body 12, for example, due to the configuration in which fastener 16 extends through only one of the first and second branches 28, 30 of the rod-receiving channel 18, although other structures may make this possible.

One of ordinary skill in the art will know and appreciate that this method is not limited to the specific order in which it is described herein, and that the procedure may be performed in whatever sequence is preferred in light of the given application and/or disorder. For example, the spinal rod 20 may be attached to the first spinal hook 10 before the second spinal fixation device is implanted, or after both the first spinal hook 10 and the second spinal fixation device are implanted. Thus, any reference herein to a specific sequence is only for ease of description.

While it is apparent that the illustrative embodiments of the invention herein disclosed fulfill the objectives stated above, it will be appreciated that numerous modifications and other embodiments may be devised by those skilled in the art. Therefore, it will be understood that the appended claims are intended to cover all such modifications and embodiments which come within the spirit and scope of the present invention.

1. A spinal fixation device comprising:
a body defining a channel for receiving a spinal rod, the channel having a first branch and a second branch separated by a lateral opening;
a hook extending from the body for engaging a portion of a vertebra; and
a fastener configured to secure the spinal rod in the channel, wherein the fastener extends through only the first branch.

2. The spinal fixation device of claim 1, wherein the fastener extends through a bore provided in the first branch.

3. The spinal fixation device of claim 2, wherein the fastener includes a head receivable in the bore and a tip opposite the head, and the tip is spaced from the second branch when the fastener secures the spinal rod in the channel.

4. The spinal fixation device of claim 2, wherein the fastener is a set screw, and at least one of the set screw and the bore is threaded.

5. The spinal fixation device of claim 1, wherein the lateral opening is configured to permit insertion of the spinal rod into the channel through the lateral opening without removing the fastener from the first branch.

6. The spinal fixation device of claim 1, wherein the fastener includes a tip that engages the spinal rod to secure the spinal rod in the channel.

7. The spinal fixation device of claim 6, wherein the tip includes a ramp surface that engages the spinal rod.

8. The spinal fixation device of claim 6, wherein the tip is substantially conical or frustoconical.

9. The spinal fixation device of claim 1, wherein the hook defines a first axis and the channel defines a second axis that is inclined with respect to the first axis by greater than about 90°.

10. The spinal fixation device of claim 9, wherein the second axis is inclined with respect to the first axis by between about 100° and about 135°.

11. The spinal fixation device of claim 1, wherein the first branch is an upper branch of the channel.

12. The spinal fixation device of claim 1, wherein the first branch is a lower branch of the channel.

13. The spinal fixation device of claim 1, further comprising at least one tool engagement recess formed on the body for gripping the body with a tool.

14. The spinal fixation device of claim 13, further comprising a first tool engagement recess formed on the body and a second engagement recess formed on the body opposite the first tool engagement recess.

15. The spinal fixation device of claim 13, wherein the at least one tool engagement recess is configured for gripping the body with a forceps.

16. The spinal fixation device of claim 15, wherein the at least one tool engagement recess is configured to allow gripping with the forceps without the forceps blocking the lateral opening.

17. The spinal fixation device of claim 13, wherein the at least one tool engagement recess is configured for gripping from above when the hook is engaged with a portion of a vertebra.

18. The spinal fixation device of claim 1, wherein the hook is configured and dimensioned for engaging a lamina.

19. A spinal fixation device comprising:
a body defining a rod-receiving channel having a lateral opening, the body including at least one tool engagement recess for gripping by a tool;
a hook extending from the body for engaging a portion of a vertebra; and
a fastener configured to secure the spinal rod in the channel.

20. The spinal fixation device of claim 19, wherein the body includes a first side and a second side opposite the first side, and a first tool engagement recess is formed on the first side and a second tool engagement recess is formed on the second side.

21. The spinal fixation device of claim 20, wherein the first and second tool engagement recesses are configured and dimensioned to engage first and second jaws of a forceps.

22. The spinal fixation device of claim 19, wherein the rod-receiving channel includes a first branch and a second branch separated by the lateral opening, and the fastener extends through only the first branch.

23. The spinal fixation device of claim 22, wherein the fastener includes a head receivable in a bore in the first branch and a tip opposite the head, and the tip is spaced from the second branch when the fastener secures the spinal rod in the rod-receiving channel.

24. The spinal fixation device of claim 22, wherein the lateral opening is configured to permit insertion of the spinal rod into the rod-receiving channel through the lateral opening without removing the fastener from the first branch.

25. The spinal fixation device of claim 22, wherein the first branch is an upper branch.

26. The spinal fixation device of claim 22, wherein the first branch is a lower branch.

27. The spinal fixation device of claim 19, wherein the fastener includes a ramp surface that engages the spinal rod.

28. The spinal fixation device of claim 27, wherein the fastener includes a tip that is substantially conical or frustoconical.

29. The spinal fixation device of claim of claim 19, wherein the fastener is a threaded set screw.

30. The spinal fixation device of claim 19, wherein the hook is configured and dimensioned for engaging a lamina.

31. The spinal fixation device of claim 19, wherein the at least one tool engagement recess is configured for gripping from above when the hook is engaged with a portion of a vertebra.

32. The spinal fixation device of claim 19, wherein the at least one tool engagement recess is configured to allow gripping with a forceps without the forceps blocking the lateral opening.

33. A method of spinal fixation comprising the steps of:
providing a first spinal fixation device having a body with an open channel for receiving a spinal rod, a hook extending from the body, and a fastener engaged with the body and capable of partially blocking the open channel;
engaging the hook to a portion of a first vertebra;
moving the spinal rod into the open channel without disengaging the fastener from the body; and
tightening the fastener against the spinal rod to secure the spinal rod in the channel.

34. The method of claim 33, wherein the step of engaging the hook to a portion of the first vertebra occurs before the step of moving the spinal rod into the open channel.

35. The method of claim 33, wherein the step of engaging the hook to a portion of the first vertebra occurs after the step of moving the spinal rod into the open channel.

36. The method of claim 33, wherein the step of moving the spinal rod into the open channel comprises moving the spinal rod into the open channel.

37. The method of claim 36, wherein the spinal rod is moved upward into the open channel at an angle of between about 20° and about 70° with respect to a medial plane of a patient.

38. The method of claim 36, wherein the spinal rod is moved upward into the open channel at an angle of between about 30° and about 60° with respect to a medial plane of a patient.

39. The method of claim 33, wherein the step of moving the spinal rod into the open channel comprises moving the open channel toward the spinal rod.

40. The method of claim 39, wherein the first spinal fixation device is moved downward onto the spinal rod at an angle of between about 20° and about 70° with respect to a medial plane of a patient.

41. The method of claim 39, wherein the first spinal fixation device is moved downward onto the spinal rod at an angle of between about 30° and about 60° with respect to a medial plane of a patient.

42. The method of claim 33, further comprising the steps of:
attaching a second spinal fixation device to a portion of a second vertebra; and
securing the spinal rod to the second spinal fixation device.

43. The method of claim 42, wherein the step of attaching the second spinal fixation device occurs before the steps of engaging the hook to a portion of the first vertebra and moving the spinal rod into the open channel.

44. The method of claim 42, wherein the step of attaching a second spinal fixation device occurs after steps of engaging the hook to a portion of the first vertebra and moving the spinal rod into the open channel.

45. The method of claim 33, further comprising the step of compressing or distracting the first vertebra with respect to the second vertebra.

46. The method of claim 33, further comprising the step of aligning the first vertebra with respect to the second vertebra.

47. The method of claim 33, wherein the open channel includes a first branch and a second branch separated by a lateral opening, and the fastener engages only one of the first branch and the second branch.

48. The method of claim 33, wherein the portion of the first vertebra is a lamina.

49. The method of claim 33, wherein the step of tightening the fastener against the spinal rod causes an inclined surface of the set screw to bias the spinal rod into the open channel.

50. The method of claim 49, wherein the inclined surface is a substantially conical or frustoconical tip of the fastener.

51. The method of claim 33, further comprising the step of removing the spinal rod from the open channel without completely disengaging the fastener from the body.

52. The method of claim 33, wherein the fastener is a threaded set screw.
